(19) United States
(12) Patent Application Publication
(10) Pub. No.: US 2022/0370324 A1
(43) Pub. Date: Nov. 24, 2022

(54) THICKENER

(71) Applicants: NATIONAL INSTITUTE OF ADVANCED INDUSTRIAL SCIENCE AND TECHNOLOGY, Tokyo (JP); KANEKA CORPORATION, Osaka-shi, Osaka (JP)

(72) Inventors: Tomohiro IMURA, Tsukuba-shi (JP); Toshiaki TAIRA, Tsukuba-shi (JP); Tadao TSUJI, Settsu-shi (JP); Satohiro YANAGISAWA, Tokyo (JP)

(73) Assignees: NATIONAL INSTITUTE OF ADVANCED INDUSTRIAL SCIENCE AND TECHNOLOGY, Tokyo (JP); KANEKA CORPORATION, Osaka-shi, Osaka (JP)

(21) Appl. No.: 17/785,170

(22) PCT Filed: Dec. 3, 2020
(86) PCT No.: PCT/JP2020/045085

§ 371 Date: Jun. 14, 2022

(30) Foreign Application Priority Data
Dec. 17, 2019 (JP) 2019-227456

(51) Int. Cl. A61K 8/64; A61Q 19/00; A61K 8/34; A61K 8/55

(52) U.S. Cl. CPC A61K 8/64; A61Q 19/00; A61K 8/345; A61K 8/553; A61K 2800/48; A61K 2800/30

(57) ABSTRACT

The objective of the present invention is to provide a thickener that can increase the viscosity of a composition with suppressing stickiness and maintaining use feeling in the case of applying the composition on the skin or the like, since the thickener does not contain a polymer thickener or the content amount of a polymer thickener is small. The thickener according to the present invention is characterized in comprising a phospholipid, a biosurfactant, and a 1,2-diol compound represented by the following formula (I):

HO—CH₂—CH(OH)—R¹ (I)

wherein R¹ is a hydrocarbon group having a carbon number of 5 or more and 12 or less.

### TECHNICAL FIELD

The present invention relates to a thickener that can increase the viscosity of a composition with suppressing stickiness and maintaining use feeling in the case of applying the composition on the skin or the like, since the thickener does not contain a polymer thickener or the content amount of a polymer thickener is small.

### BACKGROUND ART

A topical medication to be applied on the skin or the like becomes easy to be applied with suppressing dripping by increasing the viscosity when being in use in comparison with a liquid formulation such as an aqueous solution and a suspension. A thickener is therefore mixed in a topical medication to increase viscosity in some cases.

An example of a thickener generally includes a polymer such as a protein and a polysaccharide. An example of a protein thickener includes gelatin. An example of a polysaccharide thickener includes xanthane gum derived from a microorganism, carrageenan derived from seaweed, guar gum derived from a plant, and a cellulose derivative. On the one hand, a polymer thickener causes stickiness and impairs the sense of use like an adhered syrup especially after a topical medication is applied and dried. Thus, a compound and a composition that are a low molecule and have a thickening action are preferred, but a practical low molecular thickener has not ever been developed.

For example, Patent document 1 discloses a block copolymer having a hydrophobic polymer unit and a sulfonate group-containing hydrophilic polymer unit as a thickener component having a relatively low molecule weight. However, the above-described problem of stickiness cannot be solved by the block copolymer, since the molecular weight of the block copolymer is several tens of thousands or more.

### PRIOR ART DOCUMENT

### Patent Document

Patent document 1: JP H11-241060 A

### DISCLOSURE OF THE INVENTION

### Problems to be Solved by the Invention

A conventional thickener has a problem of stickiness, since a conventional thickener is a polymer such as a protein and a polysaccharide as described above.

Accordingly, the objective of the present invention is to provide a thickener that can increase the viscosity of a composition with suppressing stickiness and maintaining use feeling in the case of applying the composition on the skin or the like, since the thickener does not contain a polymer thickener or the content amount of a polymer thickener is small.

### Means for Solving the Problems

The inventors of the present invention made extensive studies to solve the above-described problem. As a result, the inventors completed the present invention by finding that a thickener can be produced by mixing the specific 1,2-diol compound in addition to a phospholipid and a biosurfactant even without using a polymer thickener.

Hereinafter, the present invention is described.
[1] A thickener comprising a phospholipid, a biosurfactant, and a 1,2-diol compound represented by the following formula (I):

HO—CH₂—CH(OH)—R¹ (I)

wherein R¹ is a hydrocarbon group having a carbon number of 5 or more and 12 or less.
[2] The thickener according to the above [1], further comprising a polyol having 3 or more hydroxy groups.
[3] The thickener according to the above [2], comprising 1 part or more by mass and 20 parts or less by mass of the polyol to 1 part by mass of the biosurfactant.
[4] The thickener according to any one of the above [1] to [3], wherein the biosurfactant is surfactin represented by the following formula (II) or a salt thereof:

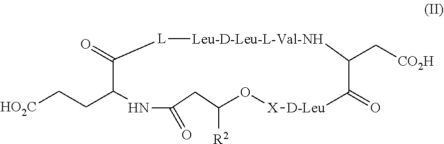

wherein X is an amino acid residue selected from leucine, isoleucine and valine, and R¹ is a C₉₋₁₈ alkyl group.
[5] A thickener,
   comprising a phospholipid, a biosurfactant and water,
   having viscosity of 30 mPa·s or more and 600 Pa·s or less,
   wherein a content amount of a polymer thickener is less than 1.0 mass %.

[6] A topical medication comprising the thickener according to any one of the above [1] to [5].
[7] The topical medication according to the above [6], wherein a content amount of a polymer thickener is less than 1.0 mass %.
[8] The topical medication according to the above [6] or [7], wherein the topical medication is a cosmetic.
[9] Use of a thickener for increasing viscosity of a composition comprising water,
   wherein the thickener comprises a phospholipid, a biosurfactant, and a 1,2-diol compound represented by the following formula (I):

HO—CH₂—CH(OH)—R¹ (I)

wherein R¹ is a hydrocarbon group having a carbon number of 5 or more and 12 or less.
[10] The use according to the above [9], wherein the thickener further comprises a polyol having 3 or more hydroxy groups.
[11] The use according to the above [10], wherein the thickener comprises 1 part or more by mass and 20 parts or less by mass of the polyol to 1 part by mass of the biosurfactant.
[12] The use according to any one of the above [9] to [11], wherein the biosurfactant is surfactin represented by the above formula (II) or a salt thereof.
[13] The use according to any one of the above [9] to [12], wherein a concentration of a polymer thickener in the composition is less than 1.0 mass %.
[14] Use of a thickener for increasing viscosity of a composition comprising water,
   wherein the thickener comprises a phospholipid, a biosurfactant and water,
   viscosity of the composition is 30 mPa·s or more and 600 Pa·s or less, and
   a content amount of a polymer thickener in the thickener is less than 1.0 mass %.

[15] A method for increasing viscosity of a composition comprising water,
the method comprising the step of mixing a phospholipid, a biosurfactant, and a 1,2-diol compound represented by the following formula (I):

HO—CH₂—CH(OH)—R¹ (I)

wherein R¹ is a hydrocarbon group having a carbon number of 5 or more and 12 or less,
in the composition.
[16] The method according to the above [15], wherein a polyol having 3 or more hydroxy groups is further mixed in the composition.
[17] The method according to the above [16], wherein 1 part or more by mass and 20 parts or less by mass of the polyol is mixed to 1 part by mass of the biosurfactant.
[18] The method according to any one of the above [15] to [17], wherein the biosurfactant is surfactin represented by the above formula (II) or a salt thereof.
[19] The method according to any one of the above [15] to [18], wherein a concentration of a polymer thickener in the composition is less than 1.0 mass %.
[20] A method for increasing viscosity of a composition comprising water,
   the method comprising the step of mixing a phospholipid, a biosurfactant, and water in the composition,
   viscosity of the thickener is 30 mPa·s or more and 600 Pa·s or less, and
   a content amount of a polymer thickener in the thickener is less than 1.0 mass %.

An example of the hydrocarbon group having a carbon number of 5 or more and 12 or less includes a C₅₋₁₂ alkyl group, a C₅₋₁₂ alkenyl group, a C₅₋₁₂ alkynyl group and a C₆₋₁₂ aromatic hydrocarbon group. The carbon number is preferably 6 or more and preferably 10 or less.

The “C₅₋₁₂ alkyl group” means a monovalent linear, branched or cyclic saturated aliphatic hydrocarbon group having a carbon number of 5 or more and 12 or less. The group is exemplified by n-pentyl, cyclopentyl, n-hexyl, cyclohexyl, n-heptyl, n-octyl, n-nonyl, n-decanyl, adamantyl, n-undecyl and n-dodecyl, preferably C₆₋₁₀ alkyl group, preferably a linear or branched alkyl group, and more preferably a linear alkyl group.

The “C₅₋₁₂ alkenyl group” means a monovalent linear, branched or cyclic unsaturated aliphatic hydrocarbon group having a carbon number of 5 or more and 12 or less and having at least one carbon-carbon double bond. The group is exemplified by n-pentenyl, n-hexenyl, n-heptenyl, cyclohexenyl, n-octenyl, n-decenyl and n-dodecenyl, preferably a C₆₋₁₀ alkenyl group, preferably a linear or branched alkenyl group, and more preferably a linear alkenyl group.

The “C₅₋₁₂ alkynyl group” means a monovalent linear, branched or cyclic unsaturated aliphatic hydrocarbon group having a carbon number of 5 or more and 12 or less and having at least one carbon-carbon triple bond. The group is exemplified by n-pentynyl, n-hexynyl, n-heptynyl, n-octynyl, n-decynyl and n-dodecynyl, preferably a C₆₋₁₀ alkynyl group, preferably a linear or branched alkynyl group, and more preferably a linear alkynyl group.

The “C₆₋₁₂ aromatic hydrocarbon group” means a monovalent aromatic hydrocarbon group having a carbon number of 6 or more and 20 or less. The group is exemplified by phenyl, naphthyl, indenyl and biphenyl, and preferably phenyl, naphthyl and biphenyl.

The “C₉₋₁₈ alkyl group” means a monovalent linear or branched saturated hydrocarbon group having a carbon number of 9 or more and 18 or less. The group is exemplified by n-nonyl, 6-methyloctyl, 7-methyloctyl, n-decyl, 8-methylnonyl, n-undecyl, 9-methyldecyl, n-dodecyl, 10-methylundecyl, n-tridecyl, 11-methyldodecyl, n-tetradecyl, n-pentadecyl, n-hexadecyl, n-heptadecyl and n-octadecyl.

### EFFECT OF THE INVENTION

The thickener of the present invention can increase the viscosity of a composition even when a polymer thickener is not used or the content amount thereof is reduced. As a result, when a composition containing the thickener of the present invention is applied to the skin or the like, the stickiness caused by a polymer thickener is suppressed particularly after the composition is dried. Thus, the thickener of the present invention is very useful as a component to increase the viscosity of a topical medication such as cosmetics.

### MODE FOR CARRYING OUT THE INVENTION

The thickener of the present invention exhibits a thickening property, even though the thickener does not contain a polymer thickener or a content amount of a polymer thickener is less than 1.0 mass %.

The molecular weight of a polymer thickener is not particularly restricted in this disclosure as long as the polymer thickener can be said to be a polymer, and for example, may be 10,000 or more. An example of a general polymer thickener includes a cellulose polymer thickener such as cellulose and a derivative thereof; an alginate polymer thickener such as sodium alginate; a starch polymer thickener such as starch, carboxymethyl starch and methyl hydroxypropyl starch; other polysaccharide polymer thickener such as agar, xanthane gum, carrageenan and guar gum; a protein polymer thickener such as pectin, collagen, casein, albumin and gelatin; a vinyl polymer thickener such as polyvinyl methyl ether and carboxy vinyl polymer; a polyoxyethylene polymer; a polyoxyethylene-polyoxypropylene copolymer; and an acrylate polymer thickener such as polyethyl acrylate and polyacrylamide.

The thickener of the present invention specifically comprises a phospholipid, a biosurfactant and water, has viscosity of 30 mPa·s or more and 600 Pa·s or less, and a content amount of a polymer thickener is less than 1.0 mass %.

For example, the viscosity is determined by rotating a circular cylinder or a circular disk in a solution that contains 0.1 mass % or more and 5 mass % or less of a phospholipid, 0.2 mass % or more and 10 mass % or less of a biosurfactant and water as a solvent and that does not contain a polymer thickener or in which the concentration of a polymer thickener is less than 1.0 mass %, measuring viscosity resistance torque acting on the used circular cylinder or circular disk with using a B-type viscometer, and converting the measured value. The viscosity is preferably 40 mPa·s or more, more preferably 50 mPa·s or more, and preferably 500 Pa·s or less.

A phospholipid is a general term for a lipid having a phosphate ester part in the structure and classified into a glycerophospholipid having a glycerin in the skeleton and a sphingophospholipid having a sphingosine in the skeleton. A sphingosine has a structure obtained by replacing the hydroxy group at the C2 position of glycerin with an amino group and further binding a long chain alkyl group at the C1 position. The phosphate group forms a phosphate ester with the hydroxy group of the glycerin or the sphingosine. A gelatinous composition and an emulsified composition containing a phospholipid are excellent in use feeling, since a phospholipid is one of constituent components of a cell membrane.

For example, the phospholipid is represented by the following general formula (III):

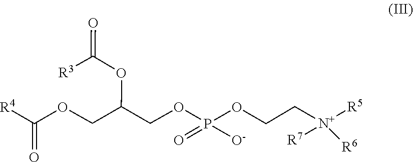

wherein R³ and R⁴ are independently a C₈₋₂₄ alkyl group, a C₈₋₂₄ alkenyl group or a C₈₋₂₄ alkynyl group, and R⁵ to R⁷ are independently a C₁₋₆ alkyl group.

The C₈₋₂₄ alkyl group means a monovalent linear or branched saturated aliphatic hydrocarbon group having a carbon number of 8 or more and 24 or less. An example of the group includes octyl, nonyl, decyl, dodecyl, tetradecyl, hexadecyl, octadecyl, eicosyl, docosyl, isotetradecyl, isohexadecyl, isooctadecyl, isoeicosyl, isodocosyl, 2-butyldecyl, 2-hexyldecyl, 2-octyldecyl, 2-decanyldecyl, 2-dodecanyldecyl and tetracosyl.

The C₈₋₂₄ alkenyl group means a monovalent linear or branched unsaturated aliphatic hydrocarbon group having a carbon number of 8 or more and 24 or less and having at least one carbon-carbon double bond.

The C₈₋₂₄ alkynyl group means a monovalent linear or branched unsaturated aliphatic hydrocarbon group having a carbon number of 8 or more and 24 or less and having at least one carbon-carbon triple bond.

The C₁₋₆ alkyl group means a monovalent linear or branched saturated aliphatic hydrocarbon group having a carbon number of 1 or more and 6 or less. An example of the group includes methyl, ethyl, n-propyl, isopropyl, n-butyl, isobutyl, s-butyl, t-butyl, n-pentyl and n-hexyl. The group is preferably a C₁₋₄ alkyl group, more preferably a C₁₋₂ alkyl group, and even more preferably methyl.

A specific example of the phospholipid includes lecithin. Lecithin has the following structure and is another name for phosphatidylcholine.

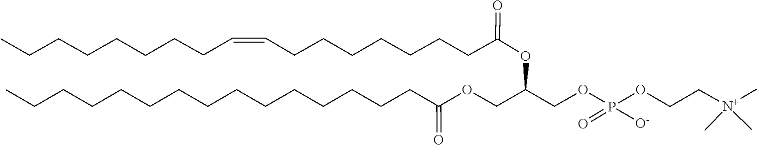 [SMILES: CCCCCCCC/C=C\CCCCCCCC(=O)O[C@H](COC(=O)CCCCCCCCCCCCCCC)COP(=O)([O-])OCC[N+](C)(C)C]

A lipid product containing a naturally derived phospholipid is sometimes called as natural lecithin. For example, egg-yolk-derived phospholipid product is sometimes called as egg-yolk lecithin, and soybean-derived phospholipid product is sometimes called as soybean lecithin.

An example of the phospholipid also includes a glycerophospholipid such as phosphatidic acid, bisphosphatidic acid, phosphatidylserine, phosphatidylethanolamine, phosphatidylmethylethanolamine, phosphatidylinositol, phosphatidylglycerol and diphosphatidylglycerol; a sphingophospholipid such as sphingosine, ceramide, sphingomyelin and cerebroside; and a hydrogenated lecithin such as hydrogenated soybean lecithin and hydrogenated egg-yolk lecithin.

A biosurfactant is a naturally derived surfactant compound produced by a microorganism or the like and exemplified by a lipopeptide biosurfactant having a hydrophobic group and a hydrophilic peptide part, especially a cyclic lipopeptide biosurfactant having a cyclic peptide part. Such a cyclic peptide part has one or more anionic groups such as a carboxy group and a phenolic hydroxy group. The lipopeptide biosurfactant has the advantage of having a small impact on the environment, since the lipopeptide biosurfactant is easily decomposed by a microorganism.

The cyclic lipopeptide biosurfactant is exemplified by one or more cyclic lipopeptide biosurfactants selected from surfactin, arthrofactin, iturin, and salts thereof, and is preferably surfactin or a salt thereof.

The surfactin or a salt thereof is the surfactin represented by the general formula (II) or a salt thereof.

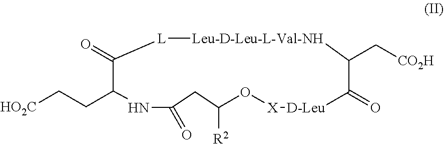

wherein R² and X are the same as the above.

The amino acid residue as the X may be any one of L-body and D-body, and is preferably L-body. An example of the counter cation that constitutes the salt of surfactin includes an alkali metal ion and a quaternary ammonium ion. The alkali metal ion is not particularly restricted, and exemplified by a lithium ion, a sodium ion and a potassium ion, and preferably a sodium ion. An example of the substituent group of the quaternary ammonium ion includes an organic group. An example of such an organic group includes a C₁₋₆ alkyl group such as methyl, ethyl, n-propyl, isopropyl, n-butyl and tert-butyl; a C₁₋₆ alkyl group having a substituent group, such as 2-hydroxyethyl; an aralkyl group such as benzyl, methylbenzyl and phenylethyl; an aryl group such as phenyl, toluyl and xylyl. An example of the quaternary ammonium ion includes a tetramethylammonium ion, a tetraethylammonium and a pyridinium ion.

Arthrofactin is represented by the general formula (IV).

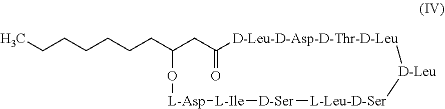

Arthrofactin has one D-aspartic acid and one L-aspartic acid respectively in the structure, and the amino acid residues may form a salt. An example of the counter cation that constitutes such a salt includes an alkali metal ion and a quaternary ammonium ion.

Iturin is represented by the general formula (V).

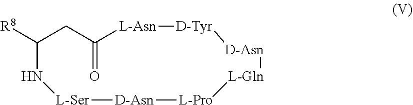

In the formula (V), R⁸ is a C₉₋₁₈ alkyl group and exemplified by —(CH₂)₁₀CH₃, —(CH₂)₈CH(CH₃)CH₂CH₃ and —(CH₂)₉CH(CH₃)₂.

One of the biosurfactant or salt thereof may be used, or two or more of the biosurfactants or salts thereof may be used. For example, the biosurfactant or salt thereof may be a mixture containing two or more of the biosurfactants or salts thereof having hydrophobic groups having different carbon numbers. The cyclic lipopeptide biosurfactant can be purified from a culture in which a microorganism that produces the target cyclic lipopeptide biosurfactant is cultivated in accordance with a publicly known method. A purified product may be used or an unpurified product may be used as the cyclic lipopeptide biosurfactant. For example, the above culture may be directly used. An example of the microorganism that produces surfactin includes a strain classified in Bacillus subtilis. The chemically synthesized biosurfactant may be similarly used.

The main solvent of the thickener according to the present invention is water, and may be water only or a mixed solvent of a water-miscible organic solvent and water. The water-miscible organic solvent means an organic solvent that can be unrestrictedly miscible with water, and preferably hardly has a negative effect on a human body, especially the skin of a human. An example of the water-miscible organic solvent includes ethanol and isopropanol. When the water-miscible organic solvent is used, a ratio of the water-miscible organic solvent to the total of water and the water-miscible organic solvent is preferably 0.1 mass % or more and 20 mass % or less, more preferably 15 mass % or less, and even more preferably 10 mass % or less or 5 mass % or less.

The thickener of the present invention exhibits a thickening property, even though the thickener does not contain a polymer thickener or a content amount thereof is small as described above. For example, the 1,2-diol compound represented by the formula (I) is mixed in addition to the phospholipid and the biosurfactant as one embodiment of the thickener according to the present invention that does not contain a polymer thickener or of which content amount of a polymer thickener is small. The 1,2-diol compound represented by the formula (I) is hereinafter described as “1,2-diol compound (I)”.

When the thickener of the present invention contains a solvent, the concentration of the 1,2-diol compound (I) may be appropriately adjusted as long as the thickener exhibits a thickening property and may be adjusted to, for example, 1 mass % or more and 20 mass % or less. When the concentration is 1 mass % or more, a thickening property is exhibited more surely even if the thickener does not contain a polymer thickener or the content amount thereof is small. On the one hand, when the concentration is 20 mass % or less, the separation of the thickener can be inhibited more surely. The ratio of the 1,2-diol compound (I) to 1 part by mass of the phospholipid may be adjusted to, for example, 5 parts or more by mass and 20 parts or less by mass. When the concentration is 5 parts or more by mass, a thickening property can be exhibited more surely even if the thickener does not contain a polymer thickener or the content amount thereof is small. On the one hand, when the concentration is 20 parts or less by mass, the separation of the thickener can be inhibited more surely.

The ratio of the 1,2-diol compound (I) to 1 part by mass of the biosurfactant may be adjusted to, for example, 2 parts or more by mass and 10 parts or less by mass. When the concentration is 2 parts or more by mass, a thickening property is exhibited more surely even if the thickener does not contain a polymer thickener or the content amount thereof is small. On the one hand, when the concentration of 10 parts or less by mass, the separation of the thickener can be inhibited more surely.

When the thickener of the present invention contains the 1,2-diol compound (I), the ratio of the biosurfactant to 1 part by mass of the phospholipid may be adjusted to, for example, 0.5 parts or more by mass and 5 parts or less by mass. The ratio of the phospholipid and the biosurfactant is included in the above range, the separation of the thickener can be inhibited more surely.

The thickener of the present invention can be produced by an ordinary method. For example, at least the phospholipid and the biosurfactant are mixed in a solvent in addition to the other component for thickening, or the thickener of the present invention may be separately mixed in a liquid composition to be thickened in order to increase the viscosity of the liquid composition. The thickener of the present invention may contain a solvent or may not contain a solvent in the latter embodiment.

The thickener and the thickened composition containing the present invention thickener according to the present invention may contain the other component in addition to the above-described phospholipid, the biosurfactant and the optional 1,2-diol compound (I) and the solvent. An example of the other component includes a polyol having 3 or more hydroxy groups, such as glycerin. The content amount of the polyol may be appropriately adjusted and may be adjusted to, for example, 1 part or more by mass and 20 parts or less by mass to 1 part by mass of the biosurfactant. The polyol having 3 or more hydroxy groups can be used as a solvent under atmospheric temperature and atmospheric pressure, and can be used as a moisturizing agent, a water retention agent, a moistening agent, a skin protective agent, an oral hygiene agent, a fragrance or the like. An example of the other component includes an ultraviolet absorber, an antioxidizing agent, an emollient agent, a solubilizing agent, an anti-inflammatory agent, a moisturizing agent, an antiseptic agent, a bactericidal agent, a dye, a fragrance and a powder.

The stickiness is suppressed and the use feeling is excellent particularly after the thickener and the thickened composition containing the present invention thickener according to the present invention are dried, since the thickener and the thickened composition do not contain a polymer thickener or the content amount thereof is small. Specifically, the concentration of a polymer thickener in the thickener or the thickened composition containing the present invention thickener according to the present invention is preferably less than 1.0 mass %. The concentration may be 0 mass %. Thus, the thickened composition containing the present invention thickener is particularly useful as a topical medication such as a cosmetic and a skin topical medication, which are directly applied to the skin.

The present application claims the benefit of the priority date of Japanese patent application No. 2019-227456 filed on Dec. 17, 2019. All of the contents of the Japanese patent application No. 2019-227456 filed on Dec. 17, 2019, are incorporated by reference herein.

### EXAMPLES

Hereinafter, the present invention is described in more detail with Examples. The present invention is however not restricted to the following Examples in any way, and it is possible to work the present invention in accordance with the Examples with an additional appropriate change within the range of the above descriptions and the following descriptions. Such a changed embodiment is also included in the technical scope of the present invention.

### Example 1

Into a 50 mL Falcon tube, 0.1 g of soybean lecithin (“BASIS LP-20” manufactured by The Nisshin OilliO Group, PC content amount: about 25%), 0.2 g of sodium surfactin and 8.7 g of 0.2% phosphate buffer solution were added. The mixture was stirred using a Vortex mixer for 2 minutes. The mixed liquid was heated to 70 to 80° C., and 1 g of 1,2-octanediol that was preliminarily heated to 70 to 80° C. was added thereto. The mixture was stirred using a Vortex mixer for 2 minutes.

### Example 2

A mixture was prepared similarly to Example 1 except that 1,2-octanediol was changed to 1,2-decanediol.

### Comparative Example 1

A mixture was prepared similarly to Example 1 except that 1,2-octanediol was changed to glycerin.

### Comparative Example 2

A mixture was prepared similarly to Example 1 except that 1,2-octanediol was changed to ethanol.

### Comparative Example 3

A mixture was prepared similarly to Example 1 except that 1,2-octanediol was changed to 1,3-propanediol.

### Comparative Example 4

A mixture was prepared similarly to Example 1 except that 1,2-octanediol was changed to 1,3-butanediol.

### Comparative Example 5

A mixture was prepared similarly to Example 1 except that 1,2-octanediol was changed to 1,2-pentanediol.

### Comparative Example 6

A mixture was prepared similarly to Example 1 except that 1,2-octanediol was changed to 1,2-hexanediol.

### Comparative Example 7

A mixture was prepared similarly to Comparative example 6 except that the amount of 1,2-octanediol was changed from 10 mass % to 40 mass %.

### Comparative Example 8

A mixture was prepared similarly to Example 1 except that 1,2-octanediol was changed to 1,8-octanediol.

### Test Example 1: Evaluation of Thickening Property

The thickening property of the mixture prepared by stirring for 2 minutes after the diol compound was added in Examples 1 and 2 and Comparative examples 1 to 8 was evaluated. Specifically, the tube containing each mixture was once reversed, reversed again, and then the thickening property was evaluated in accordance with the following criteria. The result is shown in table 1. The unit of the numerical values in Table 1 is “mass %”.

Excellent: it was apparently slower than purified water and took 1 second or more that the liquid level was horizontally returned;

Good: it was slower than purified water and took 1 second or less that the liquid level was horizontally returned;

Bad: time that elapsed before the liquid level was horizontally returned was comparable with purified water.

TABLE 1

| | | | Com- | Com- | Com- | | | | | |
|---|---|---|---|---|---|---|---|---|---|---|
| | Example | Example | parative | parative | parative | Comparative | Comparative | Comparative | Comparative | Comparative |
| | 1 | 2 | example 1 | example 2 | example 3 | example 4 | example 5 | example 6 | example 7 | example 8 |
| Soybean lecithin | 1 | 1 | 1 | 1 | 1 | 1 | 1 | 1 | 1 | 1 |
| SF | 2 | 2 | 2 | 2 | 2 | 2 | 2 | 2 | 2 | 2 |
| Glycerin | | | 10 | | | | | | | |
| Ethanol | | | | 10 | | | | | | |
| 1,3-Propanediol | | | | | 10 | | | | | |
| 1,3-Butanediol | | | | | | 10 | | | | |
| 1,2-Pentanediol | | | | | | | 10 | | | |
| 1,2-Hexanediol | | | | | | | | 10 | 40 | |
| 1,2-Octanediol | 10 | | | | | | | | | |
| 1,8-Octanediol | | | | | | | | | | 10 |
| 1,2-Decanediol | | 10 | | | | | | | | |
| 0.2% Phospate | 87 | 87 | 87 | 87 | 87 | 87 | 87 | 87 | 87 | 87 |
| buffer | | | | | | | | | | |
| Total | 100 | 100 | 100 | 100 | 100 | 100 | 100 | 100 | 100 | 100 |
| Thickening | Excellent | Excellent | Bad | Bad | Bad | Bad | Bad | Bad | Bad | Separated |
| property | | | | | | | | | | |

It was found as the result shown in Table 1 that the viscosity of a composition can be remarkably increased by mixing a surfactant-like 1,2-diol compound having a carbon number of 8 or more and having a hydrophilic part and a hydrophobic part in addition to a phospholipid and sodium surfactin. On the one hand, when 1,8-octanediol was mixed (Comparative example 8), an oil phase and an aqueous phase were separated and a homogeneous composition could not be prepared.

### Example 3

Into a 50 mL Falcon tube, 0.1 g of hydrogenated lecithin (“SLP-92H” manufactured by Tsuji Oil Mills, PC content amount: about 90%), 0.2 g of sodium surfactin and 8.7 g of 0.2% phosphate buffer solution were added. The mixture was stirred using a Vortex mixer for 2 minutes. The mixed liquid was heated to 70 to 80° C., and 1 g of 1,2-octanediol that was preliminarily heated to 70 to 80° C. was added thereto. The mixture was stirred using a Vortex mixer for 2 minutes.

### Example 4

A mixture was prepared similarly to Example 3 except that “BASIS 60HR” manufactured by The Nisshin OilliO Group and having PC content amount of about 70% was used as a hydrogenated lecithin.

### Example 5

A mixture was prepared similarly to Example 3 except that “BASIS LP-20H” manufactured by The Nisshin OilliO Group and having PC content amount of about 25% was used as a hydrogenated lecithin.

### Test Example 2: Evaluation of Thickening Property

The thickening property of the mixtures of Examples 3 to 5 was evaluated similarly to Test example 1. The result is shown in Table 2. The unit of the numerical values in Table 2 is “mass %”.

TABLE 2

| | Example 3 | Example 4 | Example 5 |
|---|---|---|---|
| Hydrogenated lecithin | 1 | | |
| (SLP-92H) PC content | | | |
| amount: about 90% | | | |
| Hydrogenated lecithin | | 1 | |
| (BASIS 60HR) PC content | | | |
| amount: about 70% | | | |
| Hydrogenated lecithin | | | 1 |
| (BASIS LP-20H) PC | | | |
| content amount: about 25% | | | |
| Sodium surfaction | 2 | 2 | 2 |
| 1,2-Octanediol | 10 | 10 | 10 |
| 0.2% Phosphate buffer | 87 | 87 | 87 |
| Total | 100 | 100 | 100 |
| Thickening property | Excellent | Excellent | Excellent |

It was found as the result shown in Table 2 that the composition can be viscous even by using a hydrogenated lecithin, not a lecithin, as the phospholipid in addition to sodium surfactin and the diol compound.

1. A thickener comprising a phospholipid, a biosurfactant, and a 1,2-diol compound represented by the following formula (I):
HO—CH₂—CH(OH)—R¹ (I)
wherein R¹ is a hydrocarbon group having a carbon number of 5 or more and 12 or less.

2. The thickener according to claim 1, further comprising a polyol having 3 or more hydroxy groups.

3. The thickener according to claim 2, comprising 1 part or more by mass and 20 parts or less by mass of the polyol to 1 part by mass of the biosurfactant.

4. The thickener according to claim 1, wherein the biosurfactant is surfactin represented by the following formula (II) or a salt thereof: 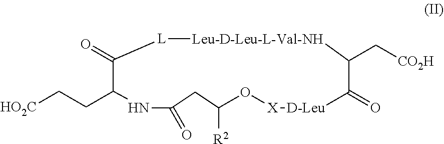
wherein X is an amino acid residue selected from leucine, isoleucine and valine, and R¹ is a C₉₋₁₈ alkyl group.

5. A thickener,
comprising a phospholipid, a biosurfactant and water,
having viscosity of 30 mPa·s or more and 600 Pa·s or less,
wherein the thickener does not comprise a polymer thickener or further comprises less than 1.0 mass % of a polymer thickener.

6. A topical medication comprising the thickener according to claim 1.

7. The topical medication according to claim 6, wherein the topical medication is a cosmetic.

8. (canceled)

9. (canceled)

10. (canceled)

11. (canceled)

12. (canceled)

13. A method for increasing viscosity of a composition comprising water, the method comprising the step of mixing a phospholipid, a biosurfactant, and a 1,2-diol compound represented by the following formula (I):
HO—CH₂—CH(OH)—R¹ (I)
wherein R¹ is a hydrocarbon group having a carbon number of 5 or more and 12 or less, in the composition.

14. The method according to claim 13, wherein a polyol having 3 or more hydroxy groups is further mixed in the composition.

15. The method according to claim 14, wherein 1 part or more by mass and 20 parts or less by mass of the polyol is mixed to 1 part by mass of the biosurfactant.

16. The method according to claim 13, wherein the biosurfactant is surfactin represented by the following formula (II) or a salt thereof: 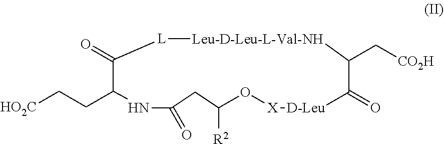
wherein X is an amino acid residue selected from leucine, isoleucine and valine, and R¹ is a C₉₋₁₈ alkyl group.

17. The method according to claim 13, wherein a concentration of a polymer thickener in the composition is less than 1.0 mass %.

18. A topical medication comprising the thickener according to claim 5.

19. The topical medication according to claim 18, wherein the topical medication is a cosmetic.
